(19) United States
(12) Patent Application Publication
(10) Pub. No.: US 2012/0329737 A1
(43) Pub. Date: Dec. 27, 2012

(54) HYBRID MOLECULES MADE OF LIPIDS AND LIPID-ANALOGOUS COMPOUNDS AND USE THEREOF AS PHARMACEUTICAL OR COSMETIC PREPARATION

(76) Inventor: Hans-Uwe Wolf, Neu-Ulm (DE)

(21) Appl. No.: 13/380,784

(22) PCT Filed: Jun. 23, 2010
(86) PCT No.: PCT/EP2010/003876

§ 371 Date: Feb. 14, 2012

Related U.S. Application Data
(60) Provisional application No. 61/219,558, filed on Jun. 23, 2009.

(30) Foreign Application Priority Data
Jul. 6, 2009 (EP) 09008828.7

(51) Int. Cl. C07C 233/18; C07H 15/06; C07C 57/00; C07J 1/00; C07C 215/24; A61P 17/00; A61K 31/685; A61K 31/7028; A61K 31/20; A61K 31/56; A61K 31/133; C07F 9/09; A61K 31/164

(52) U.S. Cl. 514/25; 554/66; 554/80; 536/18.2; 554/1; 552/502; 564/507; 514/625; 514/77; 514/558; 514/182; 514/669

(57) ABSTRACT

The invention relates to hybrid molecules made of two lipids or lipid-analogous compounds which are linked to each other via their lipophilic end. The hybrid molecules thereby have at least one hydrophilic group at their hydrophilic end for increasing the hydration shell of the hybrid molecule. The hybrid molecules according to the invention can be used as pharmaceutical or as a cosmetic preparation.

The invention relates to hybrid molecules made of two lipids or lipid-analogous compounds which are linked to each other via their lipophilic end. The hybrid molecules thereby have at least one hydrophilic group at their hydrophilic end for increasing the hydration shell of the hybrid molecule. The hybrid molecules according to the invention can be used as pharmaceutical or as a cosmetic preparation.

Basically, all biological membranes, in particular cell membranes, comprise so-called lipids and lipid-analogous substances as essential components which are structurally constructed differently but which are similar in their construction principle. The similarity in principle of the structure resides in the fact that they are constructed from a lipophilic (hydrophobic) and a hydrophilic (lipophobic) molecule part, that they hence have a so-called amphiphilic structure which is in part very greatly pronounced.

The ampiphilic structure of the lipids and lipid-analogous substances, i.e. the simultaneous presence of a (strongly) hydrophobic and of a hydrophilic, polar proportion of the molecular structure, leads to the lipids and lipid-analogous substances in an aqueous phase (generally together with other lipids) arranging themselves spontaneously to form a lipid double layer, a so-called “lipid bilayer” which represents inter alia the basis of the structure of biological membranes. The constructional principle of this bilayer is the same for all lipids and lipid-analogous substances: they are arranged in two parallel layers which are situated closely together, the hydrophobic radicals of the relevant molecules in the interior of the membrane respectively being situated directly opposite each other and coming into contact. Hence they form the hydrophobic inner region of the membrane bilayer, whilst the hydrophilic radicals are in contact on both sides of the lipid bilayer with the aqueous phase of the extra- and intracellular space. The tendency to form this lipid bilayer resides both within and outwith an organism, e.g. in an aqueous system in which the properties of the lipid bilayers can be examined in experimental arrangements designed specially for this purpose.

The group of lipids and lipid-analogous substances is composed essentially of different ceramides with a different structure (ceramide 1-9), free fatty acids (in particular palmitic acid), cholesterol and different cholesterol esters, such as e.g. cholesterol sulphate.

In the case of the ceramides, the lipophilic region consists of the alkane radical of the sphingosine basic structure and the fatty acid radical (acyl radical) coupled to the NH group of sphingosine, whilst the hydrophilic region is formed by the two OH groups and by the —NH—CO-structure of the sphingosine base body.

In the case of the phospholipids, the lipophilic region consists of the two alkane radicals of the fatty acids banded in 2- and 3-position of glycerine via an ester bond, whilst the hydrophilic region is formed by the glycerine and the phosphate group bonded in 1-position of the glycerine and also the further components bonded to the phosphate group, serine, ethanolamine, choline and inositol.

In the case of the glycolipids (cerebrosides), the lipophilic region consists of the alkane radical of the sphingosine basic structure and the alkane- or alkene radical of the fatty acid bonded to the NH₂ group, whilst the hydrophilic region is formed by the two OH groups and by the —NH—CO-structure of the sphingosine basic body and also by the monosaccharide unit bonded to the hydroxyl methylene group.

In the case of the straight-chain, unbranched saturated and unsaturated fatty acids, such as e.g. palmitic acid and linoleic acid, the hydrophobic molecular region consists of the alkane- or alkene radical of the fatty acid, whilst the hydrophilic proportion is a carboxyl group.

In the case of cholesterol and its esters and also lanosterol and sitosterol, the lipophilic part of the molecules consists of the cyclopentano-perhydro-phenanthrene basic skeleton with the branched aliphatic side chain bonded in position 17, whilst the hydrophilic structure is the OH group in position 3 of the ring system, which OH group can be esterified in addition with the strongly hydrophilic sulphate.

The structure of the lipid bilayer in an organism is formed spontaneously. Although it has significant stability, the possibility exists for example in the presence of a lipid metabolic dysfunction that a biological membrane loses a part of its lipid components because these molecules are formed either too slowly and/or in an inadequate amount or are metabolised too rapidly. As a result, the relevant membranes are depleted of the respective components, which leads inter alia to a dysfunction of the membrane structure and function.

The physiological composition of the membrane lipids of the stratum corneum of human skin is however still of essential importance for the normal structure and function of the skin for a second reason. The presence of an adequate content of these lipids ensures the unrestricted capacity of the skin to bind a physiological quantity of water. The loss of a part of the stratum corneum lipids therefore leads to a restriction in the water-binding capacity, which can be established clinically by so-called corneometry. Furthermore, in the course of the lipid loss, the so-called transepidermal water loss (TEWL) of the skin is increased. This is revealed in the occurrence of a “dry” and wrinkled skin which occurs in particular frequently, but not exclusively, with increasing age.

A known example of such changes is the depletion of ceramides in lipid bilayers of the stratum corneum of human skin. For example, in the case of atopic dermatitis, reduced contents inter alia of ceramide 3, ceramide 4, ω-hydroxy ceramides in the skin of patients (Macheleidt O, Kaiser H W and Sandhoff K (2002): J Invest Dermatol 119(1): 166-173) and sphingosine were revealed.

As numerous further scientific publications of the past years show, various skin changes and skin diseases are based on changes not only in the ceramide—but also in the overall lipid composition of the stratum corneum layer of human skin. These changes lead to a more or less greatly reduced water-binding capacity and to a restricted barrier function of the relevant skin parts for water, i.e. to an increase in the TEWL value (McIntosh T J, Stewart M E, Downing D T (1996): Biochemistry 35(12): 3,649-3,653; Coderch L, de Pera M, Perez-Cullell N, Estelrich J, de la Maza A, Parra J L (1999): Skin Pharmacol Appl Skin Physiol 12(5): 235-246). Skin changes and skin diseases of this type are for example:
atopic dermatitis
“dry” skin xerosis, xeroderma
dyshidrotic eczema
chronic cumulative toxic contact eczema
ageing skin
skin severely affected by UV light
sebostasis
hornification dysfunctions
diabetes-caused skin damage

In particular in the field of clinical medicine, it is desirable in the mentioned cases of diseases to change and/or to stabilise the structure of the biological membrane present in the organism, i.e. the lipid bilayers, in a suitable manner.

According to recent knowledge relating to the pathomechanism of atopic dermatitis (Arikawa J, Ishibashi M, Kawashima M, Takagi Y, Ichikawa Y, Imokawa G (2002): J Invest Dermatol 119(2): 433-439) and related diseases, the cause of the susceptibility of the skin in the case of such a disease is inter alia a changed lipid metabolism or reduced lipid content of the stratum corneum. These changes relate, in addition to the ceramide metabolism, inter alia, to the fatty acid metabolism. Thus there were revealed, in the case of atopic dermatitis but also in other skin diseases, such as in psoriasis, in (lamellar) ichthyosis and in contact dermatitis, reduced contents of free fatty acids in the skin of patients (Pilgrim G S, Vissers D C, van der Meulen H, Pavel S, Lavrijsen S P, Bouwstra J A, Koerten H K (2001): J Invest Dermatol 117(3): 710-717); Man M Q M, Feingold K R, Thornfeldt C R, Elias P M (1996): J Invest Dermatol 106(5): 1096-1101); Mao-Qiang M, Elias P M, Feingold K R (1993) J Clin Invest 92: 791-798; Mao-Qiang M, Jain M, Feingold K R, Elias P M (1996): J Invest Dermatol 106(1): 57-63); Velkova V, Lafleur M (2002): Chem Phys Lipids 117(1-2): 63-74.

The present-day possibilities for alleviating the symptoms and consequences of the mentioned skin diseases, in particular atopic dermatitis (there is still no talk at present of a cure), are still very limited. Topical application of special glucocorticoids and immunosuppressive active substances is associated with significant risks because of the toxicity of these substances. Specific corticoids even cause an almost counter-productive effect in that they lead to a loss of lipids, in particular of ceramides, cholesterol and free fatty acids.

Taking into account the current state of knowledge about the importance of a physiological lipid composition of the stratum corneum membranes, it is logical to attempt to compensate for deficits in membrane lipids which exist in the stratum corneum by means of exogenous supply. Therefore in practice attempts have been made to supply the missing lipids, e.g. ceramides and free fatty acids, to the changed or diseased skin with the help of ointments, creams and the like. This is effected for example by lipid preparations which are specially formulated for this purpose, inter alia by using liposomes as a vehicle for transporting lipids into the skin. Numerous products are meanwhile on the market for cosmetic purposes and for the therapy of the mentioned skin diseases.

The therapeutic measures portrayed here should of course be regarded as correct in principle since they logically attempt to compensate for the deficits existing in the stratum corneum in lipids and lipid-analogous substances. Empirical knowledge established with these therapeutic measures during the last few years reveals however that, despite the correctness in principle of the therapeutic approach, the results of these cosmetic and medicinal treatments are in no way convincing. In part, the success of the accomplished measures is uncertain or not sustainable. Even if an approximately acceptable success in the curative treatment arises, a treatment of this type has at least two serious disadvantages:
The extent of the of the successful cure is not so great that it can be called complete recovery of the diseased skin.
In order to ensure to some extent an acceptable successful cure of the skin over a fairly long period of time, the mentioned lipids and lipid-analogous substances must be supplied permanently to the skin at short time intervals, i.e. the active substances used do not show any sustained efficacy.

Both disadvantages can be attributed to a common cause. The mentioned lipids are not static components of the skin, rather they are intermediate products of a reaction sequence in which the lipids required by the skin are provided for example by synthesis processes of the organism or from nutrition and are incorporated in the biological membrane. After detection of their function as a membrane component of the stratum corneum, they are subsequently included in specific decomposition reactions of the organism.

This reaction sequence represents a steady state equilibrium in which a specific quantity of the mentioned components is synthesised by the effect of specific enzymes and is released again after a specific dwell time in the membrane from the latter in order then to be eliminated via enzyme-controlled metabolic decomposition processes. Hence a certain throughput of substance is present. The lipids supplied exogenously as skin therapeutics are included in this reaction sequence. If there is a priori a disturbance in this reaction sequence which then leads to a pathologically reduced lipid composition of the stratum corneum, then it is to be expected that the exogenous supply of lipids in the form of a therapeutic agent can fundamentally change nothing or not much in this pathological state since the exogenously supplied lipid component of the organism is metabolised in the same way as is the case with the lipid component made available endogenously.

A successful cure therefore with the therapeutic possibilities available at present is therefore dependent to a large extent upon the relevant therapeutic substitutes being able to penetrate into the skin more rapidly than they are included in the existing physiological decomposition steps and them being supplied continuously over a fairly long period of time, in extreme cases, for life.

The present problem cannot be readily resolved. Certain physiological and physical-chemical or biochemical limits are set upon the rate of absorption of lipids and lipid-analogous substances into the stratum corneum, for example with respect to the diffusion rate of the cosmetic and therapeutic active substances. This rate cannot be arbitrarily increased. On the other hand, the lipid-synthesising and lipid-decomposing enzymes involved in the mentioned reaction sequences cannot be influenced by exogenous measures or not without serious problems in the sense of increasing (synthesising enzymes) or decreasing their activity (metabolising enzymes).

The first-mentioned therapeutic approach, i.e. the activation of lipid-synthesising enzymes by exogenous active substances (e.g. nicotineamide) is possible only to a limited extent and to date has only succeeded in vitro (Tanno O, Ota Y, Kitamura N, Katsube T, Inoue S (2000): British J. Dermatol 143(3): 524-531). The second therapeutic approach, i.e. the inhibition of lipid-metabolising enzymes by exogenous active substances has obviously to date not yet been successful since obviously inhibitors of lipid-metabolising enzymes with sufficiently high specificity do not exist.

In order to resolve the described problem it is necessary basically to apply other principles in order to increase the therapeutic efficacy of exogenously supplied lipid substitute substances or analogue substances.

The object of the present invention is therefore to provide compounds by means of which
the biological membranes present in the organism can be stabilised,
the water-binding capacity of the various skin layers can be increased and
the transepidermal water loss (TEWL) of the skin can be reduced.

There should be understood by stabilisation of the biological membrane in the present case, the process that the active substances according to the invention, after incorporation in the biological membrane, display a lower tendency, relative to the unchanged original lipids, to leave the membrane, i.e. that they have greater sustainability of their cosmetic and clinical efficacy.

This object is achieved by the lipid hybrid molecules having the features of claim 1, with respect to use as pharmaceutical by the features of claim 12 or 13 and, with respect to use as cosmetic preparation, by the features of claim 15. The further dependent claims reveal advantageous developments.

According to the invention, hybrid molecules are provided made of two lipids or lipid-analogous compounds selected from the group consisting of ceramides, sphingosines, phospholipids, glycolipids, fatty acids, sterols and combinations thereof, the lipids or lipid-analogous compounds being linked to each other via their lipophilic end and, at the hydrophilic end of the lipids or lipid-analogous compounds, at least one hydrophilic group being disposed for increasing the hydration shell of the hybrid molecule.

The compounds according to the invention have the following properties:
The basic structure of the lipids or lipid-analogous substances used, which enables formation of the lipid double membrane, is not only maintained but the capacity for forming the double membrane is increased because the skin damaged by the mentioned diseases has in any case merely a restricted capacity for constructing and maintaining the physiological lipid double membrane.
The structure of the lipids or lipid-analogous substances used is changed, the basic structure being maintained, such that they can still function but only to a small extent as substrates of the metabolising enzymes present in the skin, in particular in the stratum corneum. This means that they are included to a significantly lesser extent than the original lipids or lipid-analogous substances in the respective enzymatic reaction sequences and hence they are maintained as essential structural components of the stratum corneum over a longer period of time than the original lipids or lipid-analogous substances.
The modification in the molecular structure is however effected, on the other hand, only to such a small extent that, due to the low metabolism-caused conversion or decomposition of the supplied lipid hybrid molecules, those substances are produced which are as similar as possible to the naturally occurring body lipids or lipid-analogous substances. In this way, the danger that metabolism products with a toxic effect are produced is significantly reduced.
A comparatively small, controllable decomposability of the lipid hybrid molecules is achieved in that the covalent bond between the two original lipid molecules is effected via an oxygen atom. This oxygen atom acts as metabolic predetermined breaking point since e.g. cytochrome-P₄₅₀-dependent mixed functional monooxygenases attack oxidatively the carbon atoms present in the immediate vicinity of the oxygen atom, which leads to disintegration of the hybrid molecule with formation of the ω-hydroxylated original lipids.
By bonding of a further molecule with pronounced hydrophilic properties to the hydrophilic outer molecule parts of the lipid hybrid molecules, the hydration shell of the active substances is increased, which leads consequently to an increase in the water-binding capacity of the relevant skin layers and to a reduction in the transepidermal water loss (TEWL).

These properties are thereby produced according to the invention in the following way:

By coupling two lipid molecules to form lipid hybrid molecules, it is ensured that such a molecule is decomposed or converted very much more slowly by the enzymes of the lipid metabolism present in the organism than applies to the original lipids. The molecule enlargement associated with the covalent bond between two lipid molecules leads to a great reduction in the enzymatically-controlled metabolisation because, with the known high substrate specificity of most enzymes, the (approximate) doubling in size of the lipid molecule allows the speed of the lipid conversion or lipid decomposition to be significantly reduced.

On the other hand, the resulting decomposition products are so similar in their general construction to the naturally occurring resulting products of the lipid metabolism that inclusion in the corresponding reaction sequences is possible after a slowly proceeding decomposition of the active substances according to the invention without problems. Furthermore, it need not be taken into account in any manner that the lipid hybrid molecules have relevant toxicity because of the high similarity to the original lipid molecules.

A certain degree of enzymatic metabolisation of the lipid hybrid molecules, which can be regarded however as significantly less than that of the monomeric lipid molecules, is hence a property of the molecule which is desired for pharmacokinetic and pharmacodynamic reasons because, as a result, the controllability of the therapy is better ensured than if no metabolic decomposition were possible.

A certain controlled metabolisation rate of the lipid hybrid molecules can be achieved in that an oxygen atom is present between the two coupled lipid molecules. The carbon atoms in the vicinity of this oxygen atom can be hydroxylated enzymatically, for instance by the cytochrome-P₄₅₀-dependent mixed functional monooxygenases. Such a hydroxylation taking place in the immediate vicinity of the oxygen atom leads to the formation of unstable compounds with a semiacetal structure which decompose into the corresponding reaction products. The one reaction product is a lipid molecule with ω-position OH group, the other reaction product is a lipid molecule with ca-position aldehyde function which is further oxidised to form the carboxylic acid group. It is hence obvious that, as a result of an oxidatively proceeding biochemical decomposition of the described lipid hybrid molecules, reaction products are produced which are very similar to the starting compounds of the original lipid molecules.

A further essential aspect of the pathogenesis of the above-mentioned skin changes or skin diseases is the reduced water-binding capacity of the skin tissue, in particular in the region of the stratum corneum. Physiologically, the water is incorporated not within but, since a plurality of lipid layers disposed in parallel are present, in the space between the individual lipid bilayers. This is due to the fact that the interior of the lipid bilayer is constructed from the strongly hydrophobic fatty acid esters, whilst the medium outwith the lipid bilayer is of a hydrophilic nature. Storage of water in the hydrophobic interior regions of the lipid double membrane is in practice not possible.

The initially mentioned skin changes and diseases can be attributed, on the one hand, to the loss of part of the lipids from the lipid bilayers which are disposed in parallel, on the other hand to the partial loss of water from the hydrophilic intermediate layers disposed between these bilayers. The aim of the therapeutic measures in these diseases is hence not only the reconstruction and stabilisation of the lipid bilayers themselves, as is effected with the help of the above-described lipid hybrid molecules, but in addition also the construction and stabilisation of a pronounced hydrosphere on the surface of the lipid bilayer, which are of crucial importance for the water-binding capacity of the skin.

The lipids from the group of ceramides thereby consist preferably of the compounds described previously in the scientific literature, ceramide-1 (ceramide EOS), ceramide-2 (ceramide NS), ceramide-3 (ceramide NP), ceramide-4 (ceramide EOH), ceramide-5 (ceramide AS), ceramide-6 (ceramide AP), ceramide-7 (ceramide AH), ceramide-8 (ceramide NH), and ceramide-9 (ceramide EOP) and also the corresponding ω-hydroxyceramides. However, the mentioned ceramides do not represent uniform substances with a precisely defined relative molar mass, rather each ceramide represents a family of compounds with a different length of the amide-like bonded fatty acid present in the molecule and/or the alkyl radical present in the sphingosine proportion.

The lipids from the group of sphingosine derivatives are preferably sphingosine itself and sphingomyelin which have structural similarity to the ceramides.

The lipids from the group of phospholipids are preferably phosphatidyl serine, phosphatidyl ethanolamine, phosphatidyl choline and also phosphatidyl inositol.

The lipids from the group of glycolipids are preferably the cerebrosides with the sphingosine basic skeleton and a sugar radical (glucose or galactose) and also the complex glycolipids with up to seven sugar radicals which are termed gangliosides.

The lipid-analogous substances from the group of fatty acids preferably consist of palmitic acid, stearic acid or oleic acid or of other saturated or unsaturated monocarboxylic acids with a chain length between 10 and 40 C atoms. Preferably, the fatty acids are selected from the group consisting of n-hexadecanoic acid (palmitic acid, C₁₅H₃₁—COOH), n-dodecanoic acid (lauric acid, C₁₁H₂₃—COOH), n-tetradecanoic acid (myristicinic acid, C₁₃H₂₇—COOH), n-octadecanoic acid (stearic acid C₁₇H₃₅—COOH), n-eicosanic acid (arachidic acid, C₁₉H₃₉—COOH), n-tetracosanoic acid (lignoceric acid, C₂₃H₄₇—COOH), 9-hexadecenoic acid (palmitoleic acid, C₁₅H₂₉—COOH), 9-octadecenoic acid (oleinic acid, oleic acid C₁₇H₃₃—COOH), 9,11-octadecadienoic acid (C₁₇H₃₁—COOH), 9,12-octadecadienoic acid (linoleic acid, C₁₇H₃₁—COOH), 9,12,15-octadecatrienoic acid (linolenic acid, C₁₇H₂₉—COOH), 5,8,11,14,17-eicosapentaenoic acid (“EPA”, C₁₉H₂₉—COOH), 4,7,10,13,16,19-docosahexaenoic acid (“DHA”, C₂₁H₃₁—COOH), decanoic acid (C₁₀H₂₁—COOH), octacosanoic acid (C₂₈H₅₇—COOH) and 9-octacosenoic acid (C₂₈H₅₅—COOH).

The lipid-analogous substances from the group of sterols are preferably cholesterol, cholesterol sulphate and various cholesterol esters and also further sterols such as lanesterol and sitosterol.

According to their structure, biological membranes are lipid double membranes in which the lipophilic regions of the membrane-forming lipids, inter alia also ceramides, are disposed in the interior of the membrane, as a result of which the following general structure is produced, in which the hydrophilic regions of the molecules on the membrane surface are orientated towards the extra- or the intracellular space. In FIG. 1, the arrangement of the membrane-forming lipids is represented (termed “HO-MemLipid-CH₃”).

In the general structure of the membrane lipids indicated in FIG. 1

HO-MemLipid-CH₃
the OH group characterises the hydrophilic region of the respective lipid, e.g. the OH groups contained in the sphingosine structure of the ceramides or the OH group in 3-position of the cholesterol molecule or the OH group in the carboxyl function of the fatty acids (such as e.g. palmitic acid). The indicated CH₃ group characterises the hydrophobic region of the relevant membrane lipid, e.g. the alkane- or alkene radical of the amide-like bonded fatty acid in the ceramides, the branched alkyl side chain of the cholesterol molecule or the alkane- or alkene radical of a fatty acid.

Between the lipophilic regions of the lipid molecules, relatively weak molecular attraction forces, so-called van der Waals forces, act. The forces are sufficiently great to endow the membrane with a certain stability, which can be detected inter alia by the fact that the structure of the membrane forms spontaneously. On the other hand, the forces are however not so great that they could counteract (in particular disease-induced) partial loss of lipids from the membrane.

The loss of lipids from the membrane can however be effectively reduced or prevented if not only van der Waals forces act between the lipophilic regions of specific lipid molecules but if they are linked by a covalent bond, as is indicated in the subsequent diagram 2. This diagram describes a lipid double membrane in which membrane lipids (“MemLipid”) are incorporated, which have a covalent bond respectively between the two lipophilic ends of the lipid molecules. It emerges from the schematic representation of such a linkage.

HO-MemLipid-CH₂—CH₂-MemLipid-OH
that the linkage of two identical lipid molecules to form the dimer or two different lipid molecules to form the so-called lipid hybrid molecule is effected via the two terminal CH₃ groups of the respective lipid monomers. The two OH groups of the resulting compound characterise the two hydrophilic regions at the ends of the dimer or of the lipid hybrid molecule.

Because of the (low) metabolisation capacity denoted under point 4 of the above-indicated requirements, it is recommended to insert an oxygen atom between the two lipid molecules to be bonded. Hence the following structure is produced:

HO-MemLipid-CH₂—O—CH₂-MemLipid-OH

The incorporation of such lipid hybrid molecules in the lipid double membrane produces the structure shown in FIG. 2.

Due to the covalent bond within the now present lipid hybrid molecules, the stability of this membrane structure, relative to the normal biological membrane structure, increases considerably.

In the case of ageing skin and various skin diseases, not only does a partial lipid loss of the lipid layers present in the skin occur however but there is also found a reduced water-binding capacity or an increased transepidermal water loss (TEWL) of various skin layers.

In fact, a certain quantity of water is bonded to the hydrophilic molecule parts of the membrane-position lipids, which are directed towards the intra- and extracellular space, and hence contributes to the natural water reservoir of the skin. In the case of ageing or diseased skin, this water-binding capacity obviously no longer suffices however to maintain the natural structure of the skin. It is therefore necessary to increase this water-binding capacity of the skin membranes in that in addition hydrophilic molecules are coupled to the hydrophilic molecule parts of the membrane lipids. Since, as the name already states, hydrophilic molecules have a particularly high affinity for water, the coupling of such molecules leads to an increase in the hydration shell of the membrane.

Hydrophilic groups with an increased tendency for bonding of water are strongly polar compounds, in particular with positive or negative charges and/or with those functional groups which are able to accumulate water via hydrogen bonds. There are included in particular herein
amino acids, which have strongly polar groups or charges: —COOH, —NH₂, —COO⁻ and —NH₃⁺, preferably serine, threonine, lysine, arginine, histidine, aspartic acid, glutamic acid, asparagine, glutamine, tyrosine, and tryptophan
polyols, such as ethane diol or glycerine (propanetriol) with a plurality of —OH groups in the molecule which are enabled to form hydrogen bridge bonds
sugars such as glucose or galactose in which a plurality of OH groups are present in the molecule
sugar derivatives, such as glucuronic acid or galacturonic acid with a plurality of OH groups and a dissociable —COOH group
sugar derivatives, such as amino sugar, which represent components of the strongly water-binding hyaluronic acid
organic acids, such as di- or tricarboxylic acids, malonic acid, succinic acid, malic acid or citric acid, their further carboxyl radicals not used for the bond to the lipid molecule are present dissociated and therefore charged, which accompanies a high water-binding capacity
inorganic acids, such as e.g. sulphate and phosphate which have a very high water-binding capacity due to their charges present in the molecule. Cholesterol sulphate occurs as natural component of biological membranes
choline as physiological substance with a (positively charged) quaternary N-atom
derivatives of urea: this compound (also presented with the English title “urea” in German in cosmetic and medical preparations) is, in free form, already a component of many ointments nowadays, with which an increase in water-binding capacity of the skin is intended to be effected.

The lipid molecules are preferably connected to each other covalently via their lipophilic end. The lipid molecules can thereby be connected also via a spacer. The spacer thereby consists preferably of at least one atom selected from the group consisting of carbon, nitrogen or oxygen or combinations hereof, preferably consisting of an oxygen atom or a —O—(CH₂)ₙ—O— group with n=1 to 20. It is likewise possible that the spacer comprises one of the mentioned groups.

According to the invention, the previously described dimer or the lipid hybrid molecule is likewise provided for use as pharmaceutical preparation.

According to the invention, the dimer or the lipid hybrid molecule, as were both described previously, is provided for producing a pharmaceutical for the treatment of diseases in which a dysfunction of the lipid composition of the cell membranes of an organism with respect to its content of lipids or lipid-analogous substances is present.

The previously described dimer or the lipid hybrid molecule can also be used for the production of a pharmaceutical for the treatment of diseases in which a dysfunction of the composition of the lipid bilayers of the stratum corneum of the skin with respect to its content of lipids or lipid-analogous substances is present.

A further use of the dimers according to the invention relates to the production of cosmetic preparations, in particular as cream, ointment, lotion, emulsion, gel, spray, cosmetic oil or liposomes.

If according to the invention a serine radical respectively is coupled to hydroxyl groups of the hydrophilic regions of a lipid hybrid molecule for example, then the following structure is produced:

serine-O-MemLipid-CH—O—CH₂-MemLipid-O-serine

The incorporation of a lipid hybrid molecule modified in this way in the lipid membrane (FIG. 3) leads to a significant increase in the hydrophily of the membrane surface and hence to an increase in the water-binding capacity of the membrane and consequently also of the skin.

With reference to the subsequent Figures and examples, the subject according to the invention is intended to be explained in more detail without wishing to restrict said subject to the special embodiments shown here.

FIG. 1 shows the basic construction of a biological membrane as bilayer made of membrane lipids.

FIG. 2 shows schematically the construction of a biological membrane from membrane lipid molecules and lipid hybrid molecules made of two membrane lipid components which are bonded covalently via an oxygen bridge.

FIG. 3 shows schematically the construction of a biological membrane made of membrane lipid molecules with additional incorporation of lipid hybrid molecules with two coupled serine radicals.

FIG. 4 describes an active substance in which the lipid hybrid molecule consists of ceramide 2 and cholesterol. In order to increase the hydrophily of the hydrophilic molecule areas situated on the membrane surface, a serine radical is coupled to the ceramide 2, a sulphate radical to the cholesterol.

FIG. 5 describes an active substance in which the lipid hybrid molecule consists of ceramide 3 and palmitic acid. In order to increase the hydrophily of the hydrophilic molecule areas situated on the membrane surface, a lysin radical is coupled to the ceramide 3, a urea radical to the palmitic acid.

FIG. 6 describes an active substance in which the lipid hybrid molecule consists of linoleic acid and cholesterol. In order to increase the hydrophily of the hydrophilic molecule areas situated on the membrane surface, a glucose radical is coupled to the linoleic acid, an aspartic acid radical to the cholesterol.

FIG. 7 describes an active substance in which the lipid hybrid molecule consists of ceramide 8 and lanosterol. In order to increase the hydrophily of the hydrophilic molecule areas situated on the membrane surface, a galactose radical is coupled to the ceramide 8, a phosphate radical to the lanosterol.

FIG. 8 describes an active substance in which the lipid hybrid molecule consists of cholesterol and phosphatidyl choline. In order to increase the hydrophily of the hydrophilic molecule areas situated on the membrane surface, a serine radical is coupled to the cholesterol, whilst the phosphatidyl choline remains unchanged because of its already present pronounced hydrophilic molecule part.

FIG. 9 describes an active substance in which the lipid hybrid molecule consists of palmitic acid and sphingomyelin. In order to increase the hydrophily of the hydrophilic molecule areas situated on the membrane surface, an arginine radical is coupled to the fatty acid, whilst the sphingomyelin remains unchanged because of its already present pronounced hydrophilic molecule part.

The construction of a urea-similar structure (see FIG. 5) is of particular interest for the reason that urea has a very high water-binding capacity, which is used today already in the form of urea-containing ointments for cosmetic and therapeutic treatment of those skin diseases in which drying-out of the skin represents an essential feature of the disease (e.g. in the case of dyshidrotic eczema).

The lipid hybrid molecules according to the invention can be applied in cosmetics and in medicine for therapeutic purposes wherever the natural construction of biological membranes is disrupted by pathological processes and, by using these lipid hybrid compounds, stabilisation of the membrane structure and/or a change in the membrane properties in the sense of a therapeutic goal (e.g. for increasing the membrane stability) is intended to be achieved.

Subsequently, a few examples of the therapeutic use of the lipid hybrid molecules:
According to current knowledge, skin diseases are one of the main areas for use of the mentioned lipid hybrid molecules, in particular with ceramides, saturated and unsaturated fatty acids and also cholesterol and its derivatives, not least for the reason that the mentioned lipids play a substantial role with respect to structure and function in the stratum corneum of human skin.
In the case of specific poisonings, the toxic effects of which affect preferentially the liver, such as e.g. poisoning with tetrachloromethane (tetrachlorocarbon, TETRA, CCl₄), the lipids and lipid-analogous compounds of the liver cell membranes are attacked in their structure by radicals. In this process, for example the fatty acid radicals of the lipids are oxidised, as a result of which the carbon chain is decomposed after a series of different resulting reactions. The consequence hereof is partial decomposition of the lipids and destabilisation of the membrane which leads to partial dissolution of the cell membrane and hence to severe damage to the cell. The supply of the described compounds according to the invention, e.g. fatty acid-containing lipid hybrid molecules, contributes in such a case of poisoning to a significant stabilisation of the membrane of the damaged liver cells.
A change in the lipid composition of nerve cells occurs in a large number of cases of different pathological damage to the nerve system. There are included herein inter alia neuronopathy, axonopathy and myelinopathy. There applies inter alia as causes of the damage or decomposition of the lipid-rich myelin sheaths the effect of exogenous toxic materials.
In the case of myelinopathies, such as e.g. multiple sclerosis, there are possible for stabilisation of the lipid membranes of the myelin sheaths, because of their specific structure, preferably lipid hybrid molecules, in particular with ceramides, free fatty acids and cholesterol.
In several tests, it could be detected to date that the presence of ω-3-multiple-unsaturated fatty acids in lipids has an antithrombotic effect. The background of this effect is obviously the preferred incorporation of these lipid species in the membranes of blood cells, in particular in membranes of blood plates, relative to those of lipids with ω-6-multiple-unsaturated fatty acids. The application of fatty-acid-containing hybrid molecules with a high content of ω-3-multiple-unsaturated fatty acids is possible in particular in those cases in which a genetically determined fat metabolism dysfunction leads to a high thrombotic, atherosclerotic and cardiovascular risk.

1. Lipid hybrid molecule comprising two lipids or lipid-analogous compounds selected from the group consisting of ceramides, sphingosines, phospholipids, glycolipids, fatty acids, sterols and combinations thereof, the lipids or lipid-analogous compounds being linked to each other via their lipophilic end and, at the hydrophilic end of the lipids or lipid-analogous compounds, at least one hydrophilic group being disposed for increasing the hydration shell of the hybrid molecule.

2. The lipid hybrid molecule according to claim 1, wherein the hydrophilic group is selected from the group consisting of
amino acids, in particular amino acids with polar groups or charges, preferably selected from the group consisting of —COOH, —NH₂, —COO⁻ and —NH₃⁺, preferably serine, threonine, lysine, arginine, histidine, aspartic acid, glutamic acid, asparagine, glutamine, tyrosine, and tryptophan.
polyols, in particular ethane diol or glycerine,
sugar, in particular glucose or galactose, sugar derivatives with a dissociable —COOH group, in particular glucuronic acid or galacturonic acid, or amino sugar,
organic acids, in particular di- or tricarboxylic acids, preferably malonic acid, succinic acid, malic acid or citric acid
inorganic acids, in particular sulphuric or phosphoric acids,
choline,
urea or urea derivatives
and also combinations hereof.

3. The lipid hybrid molecule according to claim 1,
wherein the bond of the hydrophilic group in the case of ceramides and glycolipids (cerebrosides) is effected either via the hydroxyl group or the hydroxymethylene group of the sphingosine base body, in the case of fatty acids via the OH group of the carboxyl grouping, in the case of sterols via the 3-position OH group, and in the case of phospholipids via the phosphate group bonded in 1-position of the glycerine.

4. The lipid hybrid molecule according to claim 3,
wherein the hybrid molecule includes in particular the following lipids or lipid-analogous compounds:
ceramide-sphingosine, ceramide-phospholipid, ceramide glycolipid, ceramide-fatty acid, ceramide-sterol, sphingosine-sphingosine, sphingosine-phospholipid, sphingosine glycolipid, sphingosine-fatty acid, sphingosine-sterol, phospholipid-phospholipid, phospholipid-glycolipid, phospholipid-fatty acid, phospholipid-sterol, glycolipid-glycolipid, glycolipid-fatty acid and glycolipid-sterol.

5. The lipid hybrid molecule according to claim 1,
wherein the ceramide molecules are selected from the group consisting of ceramide-1, ceramide-2, ceramide-3, ceramide-4, ceramide-5, ceramide-6, ceramide-7, ceramide-8, ceramide-9 and also ω-hydroxy ceramides thereof.

6. The lipid hybrid molecule according to claim 1,
wherein the lipids or lipid-analogous compounds are interconnected via their lipophilic end.

7. The lipid hybrid molecule according to claim 1,
wherein the covalent bond of the lipids or lipid-analogous compounds is effected via their lipophilic end in the case of ceramides and glycolipids (cerebrosides) either via the alkyl chain of the sphingosine basic structure or via the fatty acid radical or via the combination of both attack points, in the case of fatty acids via the terminal methyl group on the alkyl chain, in the case of sterols and their derivatives (esters) via one of the two terminal methyl groups of the branched alkyl chain at position 17 of the sterol structure and, in the case of phospholipids, via the respectively terminal methyl group of one of the two fatty acid radicals in 2-position or 3-position of glycerine or via the combination of both attack points.

8. The lipid hybrid molecule according to claim 1,
wherein the lipids or lipid-analogous compounds are bonded via a spacer.

9. The lipid hybrid molecule according to claim 8,
wherein the spacer consists of at least one atom selected from the group consisting of carbon, nitrogen or oxygen or combinations hereof or comprises these.

10. The lipid hybrid molecule according to claim 9,
wherein the spacer preferably consists of an oxygen atom or an —O—(CH₂)ₙ—O group with n=1 to 20.

11. The lipid hybrid molecule according to claim 1,
wherein the lipids or lipid-analogous compounds are selected from the group consisting of ceramides, sphingosines, phospholipids, glycolipids, fatty acids, sterols and combinations thereof.

12. A pharmaceutical preparation comprising the lipid hybrid molecule according to claim 1.

13. A method for treating diseases comprising administering the lipid hybrid molecule of claim 1.

14. The method of claim 13, wherein the disease includes a dysfunction of the composition of the lipid bilayers of the stratum corneum of the skin with respect to its content of lipids or lipid-analogous compounds.

15. A cosmetic preparation comprising the lipid hybrid molecule according to claim 1.

16. The cosmetic preparation of claim 15, wherein the cosmetic preparation is in the form of cream, ointment, lotion, emulsion, gel, spray, cosmetic oil or liposomes.

17. The method of claim 13, wherein the disease includes a dysfunction of the lipid composition of the cell membranes of an organism with respect to its content of lipids or lipid-analogous compounds.
